(19) United States
(12) Patent Application Publication
(10) Pub. No.: US 2013/0273114 A1
(43) Pub. Date: Oct. 17, 2013

(54) PHARMACEUTICAL COMPOSITIONS OF IMMUNOSUPPRESSANTS

(71) Applicants: Giancarlo Santus, Hortolandia (BR); Giuseppe Soldati, Hortolandia (BR)

(72) Inventors: Giancarlo Santus, Hortolandia (BR); Giuseppe Soldati, Hortolandia (BR)

(73) Assignee: EMS S.A., Hortolandia (BR)

(21) Appl. No.: 13/976,625

(22) PCT Filed: Jan. 18, 2012
(86) PCT No.: PCT/EP2012/000200

§ 371 Date: Jun. 27, 2013

(30) Foreign Application Priority Data
Jan. 18, 2011 (GB) 1100786.1

(51) Int. Cl. A61K 31/365; A61K 9/28

(52) U.S. Cl. CPC A61K 31/365; A61K 9/2866
USPC 424/278.1; 549/310; 424/472; 424/141.1; 530/388.1; 530/321; 546/92

(57) ABSTRACT

The present invention describes immunosuppressant pharmaceutical formulations particularly formulations of mycophenolate salts with pulsatile release behaviour.

### FIELD OF THE INVENTION

This invention relates to pulsatile release of immunosuppressants, particularly mycophenolate salts, to provide good bioavailability and reduced gastrointestinal side effects.

### BACKGROUND

Immunosuppressants are mainly used to prevent rejection in organ transplantation. They are known to have also other biological activities such as anti-inflammatory, antipsoriatic, antiviral and antitumoral activity. Immunosuppressants can function by blocking both B-and T-cells proliferation, by inhibiting inosine monophosphate dehydrogenase (IMPDH), a key enzyme involved in the purine synthesis. Immunosuppressants however have poor bioavailability.

Mycophenolic acid is a known immunosupressant but it has poor bioavailability. To overcome poor bioavailability of mycophenolic acid several attempts have been made, including the formation of ester prodrugs. U.S. Pat. No. 4,753,935 describes the most important of these esters: the morpholinoethyl ester, marketed as CELLCEPT®—ROCHE, which is absorbed and then cleaved to mycophenolic acid and achieves a remarkable improvement in bioavailability, but the technique has limitations due to side effects mainly at gastrointestinal level.

Another approach to overcome the gastrointestinal problems of mycophenolic acid that has been suggested is the development of salts of alkali metals, mainly sodium, in the form of enteric coated formulations. European Patent EP 892 640 describes, enteric coatings that remain intact for at least 2 hours in an acidic environment and then disintegrate at a pH of 6.8 that mimics intestinal juices. A limitation of this approach is in the variability linked to the different gastrointestinal pH of the patients. The most known of these preparation is marketed as Myfortic® EC tablets. This approach is described in PCT publications WO03/032978 and WO2007/093346.

A third approach is described in PCT publications WO2009/022355 and WO2006/024479, and involves extended release preparations of sodium mycophenolate where a gradual release of the active ingredient over time is obtained. In this case a limitation may be the bioavailability, as mycophenolic acid is known to have an optimal absorption window in the upper part of the intestine.

Pulsatile drug release is a known technique that delivers a burst of drug release within the body at one or more predetermined time intervals. Pulsatile release may be used to avoid drug degradation/irritation in the stomach or, for example, the ability to administer drugs released at different sites in the Gastro Intestinal tract etc. Pulsatile release can be achieved by employing tablets, capsules or pellets provided with a pH dependent or barrier membrane coating systems. Alternatively pulsatile release may be achieved by providing a water permeable membrane on a tablet, capsule, or pellet and providing a material within the tablet, capsule or pellet which expands or foams on contact with water. With this system once sufficient water has permeated the membrane the tablet, capsule or pellet will burst releasing the active drug ingredient.

U.S. Pat. No. 4,871,549 describes the “time-controlled explosion systems” in which drug release is caused by explosion of a membrane after a definite time period. This system is composed by various cores, layers, membranes and coating materials, which can be mixed in different ways in order to result in various drug release patterns. In this way sustained release preparations could be used to reduce frequency of dosing, to prevent undesirable side effects and to get optimum therapeutic efficiency.

U.S. Pat. No. 5,472,708 describes unit dosages forms for delivering drugs in a series of sequential and pulsatile release events, through a plurality of populations of coated pellets within a unit dosage form such as a capsule. By varying the proportion of the coating ingredients or coating thickness from one pellet population to another the release timing of the pellets can be controlled.

Proprietary Pulsatile Delivery Systems are also currently available from Watson Pharmaceuticals Inc (“DPHS” and “PPDS”) and from Victory Pharma Inc (“PULSYS”).

Although pulsatile release technologies are known, there is still a need for a less complex pulsatile release system which is compatible with the demands of immunosuppressants and particularly mycophenolate salts administration.

The present invention deals with a single pulse pulsatile drug delivery system obtained with a pH-independent, water insoluble but water permeable coating system applied on a swellable core containing an immunosupressant.

### SUMMARY OF THE INVENTION

One embodiment of the present invention is a pulsatile formulation of an immunosuppressant with pH-independent polymer coatings for achieving an immediate release of immunosuppressants in the upper part of the intestinal tract.

A second embodiment of the present invention is the use of the immunosuppressant pulsatile formulations to treat immunological disorders.

A third embodiment of the present invention is the process to formulate immunosuppressants with pulsatile release behaviour.

In the preferred embodiments of the invention the immunosupressant is a mycophenolate salt, particularly mycophenolate sodium salt.

### DESCRIPTION OF THE INVENTION

The present invention provides pulsatile compositions comprising an immunosupressant such as mycophenolate sodium as the active ingredient for once-a-day or twice-a-day administration wherein said composition provides a better patient treatment with good bioavailability and reduced gastrointestinal side effects.

In particular the present invention deals with single-pulse rupturable dosage forms. We have developed a dosage form with an inner compartment containing a swellable polymer and an external pH-independent, water insoluble but water permeable coating of said inner compartment. The inner compartment can be a tablet core, a pellet or a capsule.

Upon contact with gastro-intestinal fluids, water penetrates through the outer polymer coating, and/or the pores created in the film, and hydrates the swelling polymer of the inner compartment to cause it to swell.

This swelling of the polymer will develop a pressure inside the tablet, capsule or pellet which will rupture the external pH-independent coating leading to release of the drug. Before the coating rupture and release of the drug, there is a lag time controlled by the time required for the polymer of the inner compartment to swell sufficiently to cause rupture, by the permeability and mechanical properties of the external coating and by the dimensions of the pores inside the coating.

The terms “pH-independent polymer”, “pH-independent coating” refer to the properties of certain polymers, or coating formulation, not to be affected by pH. This is different for example from “enteric coating” that, is pH dependent and that remains intact in artificial gastric fluid, such as HCl pH 1, but then disintegrates in artificial intestinal fluid, such as KH₂PO4 buffered solution pH 6.8.

The presence in the inner compartment of an appropriate swellable polymer is critical for the performance of the present pulsatile formulation. In case of a film coated tablet the inner compartment is the tablet core. Swellable polymers that can be employed are derivatives of cellulose that have selective swelling characteristics and they are preferred as they swell more in basic environment than in acid one. These polymers are the so called cross-linked sodium carboxymethylcellulose (croscarmellose sodium) such as for example, Ac-Di-Sol®, Nymcel® etc. These polymers have been selected as the preferred one for their swelling characteristics.

When they are in contact with the gastric fluid the polymers start swelling and the swelling causes rupture of the pH-independent film with the release of the active ingredient.

One advantage of the preferred embodiments of this invention is that the pulsatile formulation allows good bioavailability and stability of an immunosuppressant such as mycophenolic acid without the need for enteric polymers.

Surprisingly in the preferred embodiment of the invention the innovative mycophenolic acid salt pulsatile formulation provides good stability and dissolution profile when compared with a reference product Myfortic.

Other components of the inner compartment of the tablet, capsule or pellet can be diluents such as lactose, mannitol, starch, microcrystalline cellulose, etc. and lubricants and glidants such as magnesium stearate, talc, colloidal silicon dioxide, binders such as povidone, modified starch, PVA etc. Examples of materials that can be employed are reported in Remington “The science and practice of Pharmacy” 21st edition, 2005 and in the “Handbook of Pharmaceutical Excipients” 2006, 5th edition.

The inner compartment is coated with pH-independent polymer coatings such as ethylcellulose e.g. ETHOCEL® from Dow, AQUACOAT® from FMC, or SURELEASE® from Colorcon. The coating can be applied in aqueous dispersion or in organic solvents. The amount of coating comprises 5 to 50% and more preferably 8-20% of the total weight of the composition. The coating may further include one or more components such as pore formers, plasticizers, wetting agents, etc. Example of pore former is hydroxypropylmethylcellulose (HPMC) e.g. METHOCEL®, polyethylenglycol, polyvinylpyrrolidone, etc.

Suitable plasticizers are triethyl citrate, dibutylsebacate, triacetine etc. The plasticizer is present in amount 1 to 40% by weight and preferably 5-10% of the total weight of the coating.

The present invention may be used for the improved bioavailability of any immunosuppressant. Although it has been described in relation to the preferred immunosuppressant sodium mycophenolate it can be used with immunosuppressants such as cyclosporin, tacrolimus, voclosporin, monoclonal antibiotics and rapamycin or mixtures thereof.

The preferred sodium mycophenolate compositions of the invention are administered at a dose in the range of 0.5 to 2.0 g/day of sodium mycophenolate. A common dosage and administration is about 1.5 g/day divided twice daily. The recommended dose in adult renal transplantation is 720 mg twice daily (BID), preferably on an empty stomach, one hour before or two hours after food intake. In paediatric patients the dose must be adjusted according to body surface area, the dose being of 400 mg/m² up to a maximum of 720 mg BID.

The sodium mycophenolate compositions of the present invention can be administered in combination with other immunosuppressant such as for example conventional steroids, cyclosporine, tacrolimus, sirolimus, monoclonal antibodies, etc. The sodium mycophenolate compositions can be given in concomitant, sequential or separate administration with the other immunosuppressants.

The present invention will now be illustrated by the following examples. It is understood, however, that such examples are provided for illustration only, and the invention is not intended to be limited by the examples. The formulation based on the system employed in the examples can be formed by any suitable method known in the art.

### EXAMPLE 1

### Preparation of Pulsatile Mycophenolate Formulation

### Composition of the Core

| | |
|---|---|
| Sodium mycophenolate | 384.8 mg |
| (corresponding to Mycophenolic acid 360 mg) | |
| Lactose anhydrous | 110.5 mg |
| Croscarmellose sodium | 65.0 mg |
| Povidone K30 | 40.0 mg |
| Colloidal silicon dioxide | 13.2 mg |
| Magnesium stearate | 6.5 mg |
| Ethanol (removed during the process) | 50.0 mg |

### Composition of the Film

| | |
|---|---|
| Ethylcellulose | 9.75 mg |
| Hypromellose 5 cps | 4.5 mg |
| Triethylcitrate | 0.75 mg |
| Acetone/water (removed during the process) | 300.0 mg |

### Procedure

Mycophenolate sodium, colloidal silicon dioxide and povidone were sieved and blended for a few minutes.

The mixture of powders was kneaded with ethanol and the wet granulate was passed through a 2.0 mm sieve.

The granulate was dried in a static oven at 40-50° C. and then passed through a 0.8 mm sieve.

Further excipients (lactose, croscarmellose sodium and magnesium stearate) were then added.

After blending, the final mixture was compressed to the target weight (17.0×7.2 mm capsule shaped concave punches).

The core tablets obtained were coated in a pan coater using a solution of ethylcellulose in acetone:water (90:10 w/w). Tablets bed temperature: 30-35° C.

### EXAMPLE 2

### Preparation of Pulsatile Mycophenolate Formulation With Aqueous Film Coating

Core

| | |
|---|---|
| Sodium mycophenolate | 384.8 mg |
| (corresponding to Mycophenolic acid 360 mg) | |
| Lactose anhydrous | 110.5 mg |
| Croscarmellose sodium | 80.0 mg |
| Povidone K30 | 25.0 mg |
| Colloidal silicon dioxide | 13.2 mg |
| Magnesium stearate | 6.5 mg |
| Ethanol (removed during the process) | 45.0 mg |

### Composition of the Aqueous Film

Dry film (8% total of the film)

| | |
|---|---|
| Ethylcellulose dispersion Surelease ® | 80% |
| Hypromellose (5 cps) | 15% |
| Triethylcitrate | 5% |
| Purified Water (92% total of the film) | |

The core tablets of Example 2, were prepared with the same manufacturing method reported in the Example 1, and were then coated in a pan coater using an aqueous dispersion of ethylcellulose.

The film-coating suspension was maintained under gentle agitation during the process. Tablets bed temperature: 45° C.

Samples of tablets were collected at different times of spraying equivalent at 1 mg; 3 mg; 5 mg; 10 mg and 15 mg of dry film and the dissolution was measured and it is reported in Example 4 point B.

### EXAMPLE 3

### Preparation of Pulsatile Formulation With Coating Without Plasticizer Triethylcitrate

### Composition of the Core

The core was prepared as in Example 2

### Composition of the Film

Dry film (8% total of the film)

| | |
|---|---|
| Ethylcellulose dispersion Surelease ® | 85% |
| Hypromellose (5 cps) | 15% |
| Purified Water (92% total of the film) | |

The core tablets of Example 3 were coated in a pan coater using the aqueous dispersion of ethylcellulose.

The film-coating suspension was maintained under gentle agitation during the process. Tablets bed temperature: 45° C.

Samples of tablets were collected at different times of spraying equivalent at 5 mg and 10 mg of dry film.

### EXAMPLE 4

### In vitro Release

The dissolution test method applied was the FDA official method for mycophenolic acid delayed release tablets: USP apparatus II (paddle); 50 rpm; Medium: Acid stage: 0.1N HCl (120 min), Buffer stage: buffer solution pH 6.8 (60 min); Volume 750 ml (acid), 1000 ml (buffer); Sampling time (min) 120 (acid) 10, 20, 30, 45 and 60 (buffer).

### A) Dissolution Test Performed on Film Coated Tablets of Formulation 1 vs. the Reference Myfortic EC.

| | Form. Example 1 | Myfortic EC |
|---|---|---|
| | (n = 6) | (n = 12) |
| Sampling times (min) | % Released | % Released |
| 0 | 0 | 0 |

| | | | |
|---|---|---|---|
| 120 | (acid stage) | 0 | 0 |
| 120 + 10 | (buffer stage) | 4 | 1 |
| 120 + 20 | (″) | 30 | 22 |
| 120 + 30 | (″) | 56 | 58 |
| 120 + 45 | (″) | 89 | 95 |
| 120 + 60 | (″) | 98 | 102 |

The two release profiles are similar.

### B) Dissolution Test Was Performed on Aqueous Film Coated Tablets of Example 2 at Different Amount of Coating.

The results obtained are reported below:

| | | % released at different amount of coating | | | | |
|---|---|---|---|---|---|---|
| Sampling times (min) | | 1 mg | 3 mg | 5 mg | 10 mg | 15 mg |
| 0 | | 0 | 0 | 0 | 0 | 0 |
| 120 | (acid stage) | 2 | 1 | 2 | 2 | 0 |
| 120 + 10 | (buffer stage) | 29 | 23 | 14 | 10 | 5 |
| 120 + 20 | (″) | 64 | 52 | 32 | 25 | 12 |
| 120 + 30 | (″) | 88 | 73 | 52 | 49 | 18 |
| 120 + 45 | (″) | 97 | 94 | 75 | 76 | 31 |
| 120 + 60 | (″) | 101 | 102 | 89 | 90 | 44 |

As expected the release of mycophenolate decreases, as the amount of core coating increases.

C) Dissolution Test Was Performed on Aqueous Film Coated Tablets of Example 3 at Different Amount of coating Without Plasticizer.

The results obtained are reported below:

| | | % released at different amount of coating | |
|---|---|---|---|
| Sampling times (min) | | 5 mg | 10 mg |
| 0 | | 0 | 0 |
| 120 | (acid stage) | 2 | 1 |
| 120 + 10 | (buffer stage) | 21 | 12 |
| 120 + 20 | (″) | 53 | 25 |
| 120 + 30 | (″) | 82 | 38 |
| 120 + 45 | (″) | 94 | 58 |
| 120 + 60 | (″) | 100 | 77 |

As expected the release of mycophenolate decreases, increasing the amount of coating on the core.

### EXAMPLE 5

### Stability

The stability of the formulations prepared as described in Examples 1 to 3 was checked for assay, related substances and dissolution after 6 months at room temperature and in accelerated conditions, according to ICH guidelines, and there were no significant changes from initial results.

EXAMPLE 6

### Biological Activity

The biological activity of the compositions of the present invention vs. the reference commercial product Myfortic® can be assessed in standard clinical bioequivalence trials. For example, two tablets of 384.8 mg of sodium mycophenolate (equivalent to 360 mg mycophenolic acid) of Myfortic® and of the example 1 of the present invention may be administered to 24 stable renal transplant patients in a single dose, in an open label two-way cross over design, as reported by Arns W et al. in Clin Transplant 19, 199-206 (2005). Reduction in variability of AUC, Cₘₐₓ and Tₘₐₓ of Example 1 can be expected.

1. A pulsatile release pharmaceutical composition wherein the active ingredient is an immunosupressant.

2. A pulsatile release composition according to claim 1 in which the active ingredient is a mycophenolate salt.

3. A pulsatile release pharmaceutical composition according to claim 1 comprising a compartment containing the immunosuppressant and at least a water swellable polymer, said compartment being coated with a pH-independent polymer and having, optionally, a pore forming substance.

4. A composition according to claim 2 wherein the mycophenolate salt is present in amount between 20 and 80% wfw of the formulation.

5. A composition according to claim 2 wherein the compartment is a tablet, a capsule or a pellet.

6. A composition according to claim 3 wherein the water swellable polymer is croscarmellose sodium.

7. A composition according to claim 3 wherein said pH-independent polymer is ethylcellulose.

8. A composition according to claim 3 wherein the pore forming substance is a water soluble compound.

9. A composition according to claim 8 wherein said water soluble compound is hydroxypropylmethyl cellulose.

10. A composition according to claim 1 in which the immunosuppressant is selected from the group consisting of cyclosporin, tacrolimus, voclosporin, rampamycin and monoclonal antibodies.

11. A composition according to claim 1 in which the immunosuppressant is a mixture of a mycophenolate salt and an immunosupressant selected from the group consisting of cyclosporin, tacrolimus, voclosporin, rampamycin and monoclonal antibodies.

12. A method comprising use of the composition of claim 1 for immunosuppressing a subject in need of immunosuppression.

13. A method according to claim 12 wherein the administration is with the concomitant, sequential or separate administration of another immunosuppressant.

14. A process for the preparation of a pulsatile release pharmaceutical composition according to claim 1 comprising:
(i) mixing an immunosuppressant and pharmaceutically acceptable additives,
(ii) granulating the mixture of step (i)
(iii) compressing the granulation of step (ii)
(iv) coating the tablets of step (iii) with a pH-independent coating.

15. A process according to claim 14 in which the immunosuppressant is a mycophenolate salt.

16. A process according to claim 15 in which the salt is a sodium salt.
